(19) United States
(12) Patent Application Publication
(10) Pub. No.: US 2025/0152670 A1
(43) Pub. Date: May 15, 2025

(54) METHOD FOR ALLEVIATING PULMONARY FIBROSIS USING EPIDERMAL GROWTH FACTOR

(71) Applicants: WENZHOU PRARUCOM BIO-CHEMICAL TECHNOLOGY CO., Wenzhou CIty, Zhejiang (CN); PUHSIN LTD., Kaohsiung City (TW)

(72) Inventors: Le-Shin CHANG, Kaohsiung City (TW); Shang-Yu CHIA, Kaohsiung City (TW)

(21) Appl. No.: 18/839,688

(22) PCT Filed: Apr. 28, 2022
(86) PCT No.: PCT/CN2022/089715

§ 371 Date: Aug. 19, 2024

(51) Int. Cl. A61K 38/18; A61K 9/00; A61K 31/722; A61K 31/728; A61P 11/00

(52) U.S. Cl. CPC A61K 38/1808; A61K 9/0078; A61K 31/722; A61K 31/728; A61P 11/00

(57) ABSTRACT

A method for alleviating pulmonary fibrosis includes administering to a subject in need thereof a composition containing epidermal growth factor. The composition further contains a polysaccharide and hyaluronic acid. Epidermal growth factor, the polysaccharide, and hyaluronic acid are present in a weight ratio ranging from 1:0.5:12.5 to 1:250:1250. Epidermal growth factor can exhibit satisfactory efficacy in alleviating pulmonary fibrosis and PF-related tissue lesions, and this efficacy can be enhanced when epidermal growth factor is used in combination with the polysaccharide and hyaluronic acid.

### FIELD

The present disclosure relates to a method for alleviating pulmonary fibrosis using epidermal growth factor.

### BACKGROUND

Pulmonary fibrosis (PF) is caused by excessive deposition of extracellular matrix (ECM) (especially collagen) in the lung tissue during wound healing. Diet, physiological metabolism, viruses, toxins, genetics, and immune diseases may contribute to pulmonary fibrosis. The main symptoms of pulmonary fibrosis include cough, low blood oxygen level, shortness of breath, and breathing difficulty. In severe cases, pulmonary fibrosis may lead to respiratory failure and even death.

Previous studies have demonstrated a correlation between epidermal growth factor receptor (EGFR) signaling pathway and pulmonary fibrosis. For example, as reported in Venkataraman T. et al. (2017), J. Virol., 91 (12): e00182-17, in mouse models of severe acute respiratory syndrome coronavirus (SARS-COV) pathogenesis, the wound repair pathway controlled by the epidermal growth factor receptor (EGFR) is critical to recovery from SARS-COV-induced tissue damage. In mice with constitutively active EGFR (EGFR (DSK5) mice), SARS-COV infection causes enhanced lung disease. In addition, during infection, the EGFR ligands, i.e., amphiregulin (AREG) and heparin-binding EGF-like growth factor (HB-EGF), are upregulated, and exogenous production of these ligands during infection leads to enhanced lung disease and altered wound healing dynamics. The upregulation of EGFR could produce an increased but uncontrolled wound healing, leading to fibroblast proliferation and fibrosis.

In addition, as reported in Vagapova E. R et al. (2021), Sci. Rep., 11:11234, EGF and EGF-inducible genes are involved in SARS-COV-2-fibrosis and inflammation, and such SARS-COV-2-fibrosis could be prevented by targeting EGFR/MAPK pathway with EGFR/ErbB inhibitors (such as gefitinib and dasatinib).

In spite of the aforesaid, there is still a need to develop an effective way for alleviating pulmonary fibrosis.

### SUMMARY

Accordingly, in a first aspect, the present disclosure provides a composition for alleviating pulmonary fibrosis, which can alleviate at least one of the drawbacks of the prior art, and which includes epidermal growth factor.

In a second aspect, the present disclosure provides a method for alleviating pulmonary fibrosis, which can alleviate at least one of the drawbacks of the prior art, and which includes administering to a subject in need thereof the aforesaid composition.

In a third aspect, the present disclosure provides use of the aforesaid composition in the manufacture of a medicament or a food product for alleviating pulmonary fibrosis in a subject. Such use can alleviate at least one of the drawbacks of the prior art.

### BRIEF DESCRIPTION OF THE DRAWINGS

Other features and advantages of the present disclosure will become apparent in the following detailed description of the embodiments with reference to the accompanying drawings, of which:

FIG. 1 shows the Penh index determined in each group of Example 1, infra, in which the symbols “**”, “***”, and “****” respectively represent p<0.01, p<0.001, and p<0.0001 (compared with the pathological control group);

FIG. 2 shows the scale of tissue lesion determined in each group of Example 1, infra, in which the symbols “**” and “****” respectively represent p<0.01 and p<0.0001 (compared with the pathological control group); and

FIG. 3 shows the scale of pulmonary fibrosis determined in each group of Example 1, infra, in which the symbols “*”, “**”, and “****” respectively represent p<0.05, p<0.01, and p<0.0001 (compared with the pathological control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined all technical otherwise, and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of methods for alleviating pulmonary fibrosis, the applicant surprisingly found that, EGF can exhibit satisfactory efficacy in alleviating pulmonary fibrosis (PF) and PF-related tissue lesions, and this efficacy can be enhanced when EGF is used in combination with chitosan and HA.

The present disclosure provides a composition for alleviating pulmonary fibrosis, which includes epidermal growth factor (EGF).

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

According to the present disclosure, the epidermal growth factor suitable for use in this disclosure may be derived from humans or other animals, plants, and microorganisms, and may be obtained as commercial products, or may be prepared using techniques well-known to those skilled in the art. For example, the epidermal growth factor may be a natural product isolated from a biological material (such as human tissues), or a recombinant protein or a functional fragment thereof obtained by genetic engineering. In certain embodiments, the epidermal growth factor is recombinant human epidermal growth factor (rhEGF).

According to the present disclosure, the composition may further contain a polysaccharide and hyaluronic acid (HA).

In certain embodiments, the epidermal growth factor, the polysaccharide, and hyaluronic acid in the composition are present in a weight ratio ranging from 1:0.5:12.5 to 1:250:1250. In an exemplary embodiment, the epidermal growth factor, the polysaccharide, and hyaluronic acid are present in a weight ratio of 1:5:125.

According to the present disclosure, the polysaccharide may be selected from the group consisting of chitosan, chitin, glycosaminoglycan, cellulose, starch, peptidoglycan, and combinations thereof. In an exemplary embodiment, the polysaccharide is chitosan.

According to the present disclosure, the composition may be formulated as a food product using a standard technique well known to one of ordinary skill in the art. For example, the composition may be directly added to an edible material or may be used to prepare an intermediate composition (e.g., a food additive or a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a dosage form suitable for oral administration, parenteral administration, topical administration, or respiratory tract administration using technology well known to those skilled in the art.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intrasynovial injection, intraarticular injection, intrathecal injection, intracranial injection, intraepidermal injection, subcutaneous injection, intradermal injection, intralesional injection, and sublingual administration. In certain embodiments, the pharmaceutical composition may be administered via intraperitoneal injection, intravenous injection, or sublingual administration.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

According to the present disclosure, the pharmaceutical composition may be formulated into a spray (e.g., a nasal spray or an oral spray) suitable for oral inhalation or nasal inhalation. In certain embodiments, the pharmaceutical composition may be formulated into a spray suitable for nasal inhalation.

According the to present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

The present disclosure provides a method for alleviating pulmonary fibrosis, which includes administering to a subject in need thereof the aforesaid composition.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

The present disclosure also provides use of the aforesaid composition in the manufacture of a medicament or a food product for alleviating pulmonary fibrosis in a subject.

The dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### Examples

### General Experimental Materials:

1. Epidermal growth factor (EGF) (Cat. No. 02-108), chitosan (Cat. No. 04-121), and hyaluronic acid (HA) (Cat. No. 07-113) used in the following experiments were purchased from JOYCOM BIO-CHEM CO., LTD, Taiwan.

### General Procedures:

### 1. Statistical Analysis

All the experiments described below were performed in triplicates. The experimental data are expressed as mean±standard deviation (SD). Statistical analysis was conducted using GraphPad Prism 9.0 (GraphPad Software, San Diego, USA). All the data were analyzed using two-way analysis of variance (ANOVA) followed by Dunnett's post hoc test, so as to evaluate the differences between the groups. Statistical significance is indicated by p<0.05.

### Example 1. Evaluation for the Effect of EGF on Alleviating Pulmonary Fibrosis (PF)

### A. Preparation of Mixture Containing EGF, Chitosan, and HA

EGF, chitosan, and HA were mixed in a weight ratio of 1:5:125 in sterile water, so as to obtain a mixture containing EGF (20 μg/g), chitosan, and HA.

### B. Experimental Mice

Male C57BL/6 mice (6 weeks old, with a body weight of approximately 16 to 20 g) used in the following experiments were purchased from National Laboratory Animal Center, Taipei City, Taiwan. All the experimental mice were housed in an animal room under the following laboratory conditions: an alternating 12-hour light and 12-hour dark cycle, a temperature maintained at 21° C. to 24° C., and a relative humidity maintained at 45% to 70%. Furthermore, water and feed were provided ad libitum for all the experimental mice. All experimental procedures involving the experimental mice were in compliance with the legal provision of the Institutional Animal Care and Use Committee of National Chung Hsing University, Taiwan, and were carried out according to the Guide for the Care and Use of Laboratory Animals of National Institutes of Health (NIH).

### C. Induction of PF and Administration of EGF

The C57BL/6 mice were divided into six groups, including one normal control group, one pathological control group, and four experimental groups (i.e., experimental groups 1 to 4) (n=8 mice in each group).

A respective one of EGF and the mixture containing EGF, chitosan, and HA was dissolved in phosphate-buffered saline (PBS), so as to obtain an EGF solution and a mixture solution. The mice in the experimental groups 1 and 2 were administered with the EGF solution and the mixture solution, respectively, via nasal inhalation using a nebulizer (PARI, BOY N) for 30 minutes. The mice in the experimental groups 3 and 4 were administered with EGF solution the the and mixture solution, respectively, via intraperitoneal injection. The mice of the pathological control group were administered with PBS via nasal inhalation using a nebulizer (PARI, BOY N) for 30 minutes. Each mouse was administered once daily for a total period of 19 days. In addition, the mice of the normal control group received no treatment.

The treating agent and the dose of EGF for each group are summarized in Table 1 below.

TABLE 1

| | | Dose of EGF |
|---|---|---|
| Group | Treating agent | (μg/kg) |
| Normal control group | — | 0 |
| Pathological control | PBS | 0 |
| group | | |
| Experimental group 1 | EGF solution | 330 |
| Experimental group 2 | Mixture | 330 |
| | solution | |
| Experimental group 3 | EGF solution | 30 |
| Experimental group 4 | Mixture | 30 |
| | solution | |

On the first day, after treatment with the treating agent, the mice in each of the pathological control group and experimental groups 1 to 4 were intratracheally instilled with bleomycin (BLM) at a dose of 2 mg/kg, so as to induce PF. In addition, the mice of the normal control group received no treatment. 10

### D. Determination of Lung Function

On the 15^{th} day, after treatment with the treating agent, the mice in each group were treated with methacholine to induce airflow obstruction, followed by conducting airway responsiveness measurement using Buxco FinePointe whole body plethysmography (DSI Buxco). Briefly, each mouse was subjected to inhalation of aerosolized PBS, followed by determining the Penh value as a baseline. Next, each mouse was subjected to inhalation of aerosolized methacholine (Sigma-Aldrich, Cat. No. A2251-25G) (at concentrations of 0 mg/mL, 6.25 mg/mL, 12.5 mg/mL, 25 mg/mL, and 50 mg/mL), followed by determining the increase in Penh value as a Penh index. The lower the Penh index, the better the respiratory function is.

The data thus obtained were analyzed according to the method described in section 1 of the General Procedures.

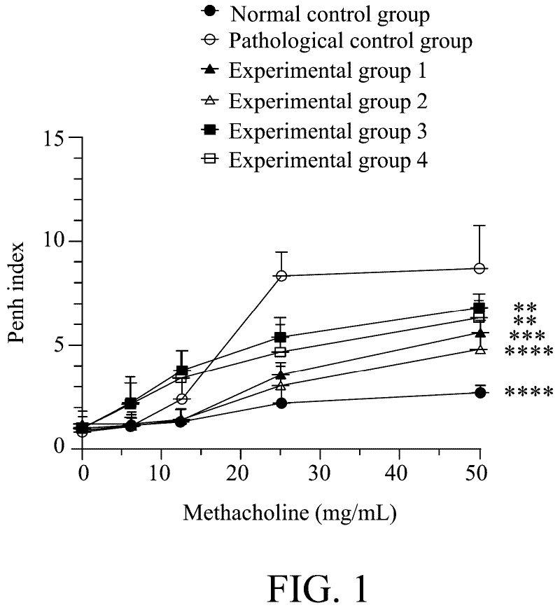

Referring to FIG. 1, the Penh indexes determined in the experimental groups 1 to 4 were each significantly lower than that determined in the pathological control group. In particular, the Penh index determined in the experimental group 2 was lower than that determined in the experimental group 1, and the Penh index determined in the experimental group 4 was lower than that determined in the experimental group 3.

These results indicate that regardless of the route of administration, i.e., nasal inhalation or intraperitoneal injection, EGF can exhibit satisfactory efficacy in alleviating pulmonary fibrosis, and this efficacy can be enhanced when EGF is used in combination with chitosan and HA.

### E. Histopathologic Analysis

After the 19th day of treatment, the respective mouse was sacrificed, and the lung tissue was obtained from the respective mouse carcass. The lung tissue was subjected to a fixation treatment with a 10% neutral buffered formalin (BiOTnA Biotech, Cat. No. TABS06-4000) at room temperature for 24 hours. The fixed tissue sample was then embedded with paraffin, followed by slicing to obtain a tissue section having a thickness of 5 μm.

The tissue section was subjected to hematoxylin-eosin staining and Masson's trichrome staining using a staining protocol well-known to those skilled in the art, and was then observed under an optical microscope (Olympus, CKX41) at a magnification of 400×. One area of the respective tissue section was randomly selected and photographed, and the pathological change in the respective tissue section was assessed according to the methods described in Schniering J. et al. (2018), Arthritis Res. Ther., 20:183 and Ruscitti F. et al. (2020), Front. Pharmacol., 11:1117. The degrees of tissue lesion and pulmonary fibrosis were ranked by scoring on a scale from 1 to 5. The higher the scale, the higher the severity of tissue lesion and pulmonary fibrosis is.

The data thus obtained were analyzed according to the method described in section 1 of the General Procedures.

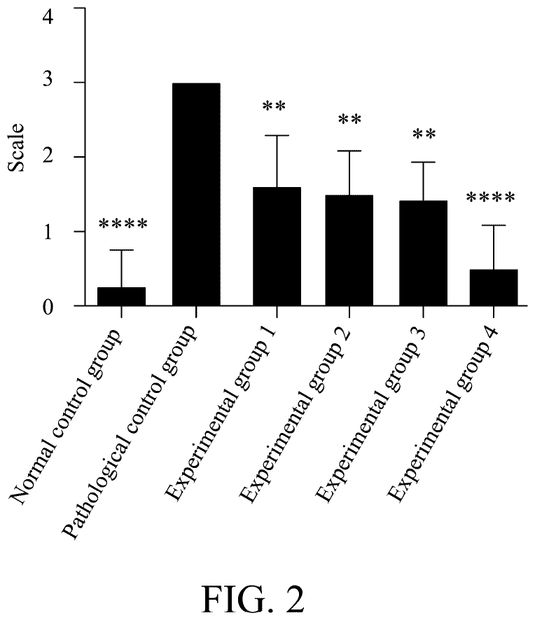

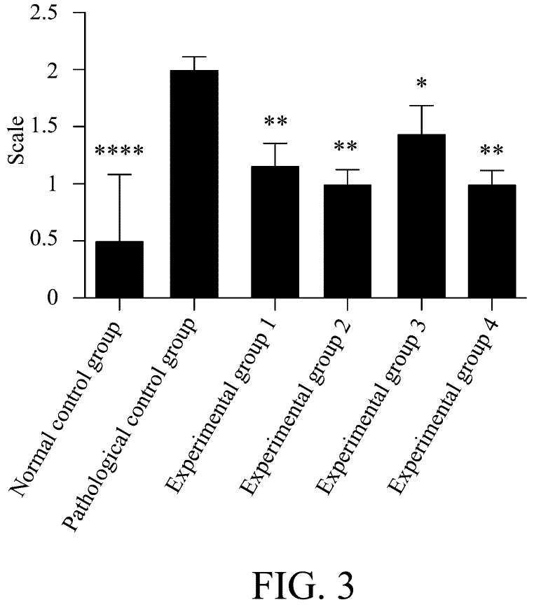

FIGS. 2 and 3 respectively show the scoring results of tissue lesions and pulmonary fibrosis. As shown in FIGS. 2 and 3, the scales of tissue lesion and pulmonary fibrosis determined in the experimental groups 1 to 4 were each significantly lower than those determined in the pathological control group. In particular, the scales of tissue lesion and pulmonary fibrosis determined in the experimental group 2 were lower than those determined in the experimental group 1, and the scales of tissue lesion and pulmonary fibrosis determined in the experimental group 4 were significantly lower than those determined in the experimental group 3.

These results indicate that regardless of the route of administration, i.e., nasal inhalation or intraperitoneal injection, EGF can exhibit satisfactory efficacy in alleviating pulmonary fibrosis and PF-related tissue lesions, and this efficacy can be enhanced when EGF is used in combination with chitosan and HA.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

1. A method for alleviating pulmonary fibrosis, comprising administering to a subject in need thereof a composition containing epidermal growth factor.

2. The method as claimed in claim 1, wherein the composition further contains a polysaccharide and hyaluronic acid.

3. The method as claimed in claim 2, wherein epidermal growth factor, the polysaccharide, and hyaluronic acid are present in a weight ratio ranging from 1:0.5:12.5 to 1:250:1250.

4. The method as claimed in claim 3, wherein epidermal growth factor, the polysaccharide, and hyaluronic acid are present in a weight ratio of 1:5:125.

5. The method as claimed in claim 2, wherein the polysaccharide is selected from the group consisting of chitosan, chitin, glycosaminoglycan, cellulose, starch, peptidoglycan, and combinations thereof.

6. The method as claimed in claim 5, wherein the polysaccharide is chitosan.

7. The method as claimed in claim 1, wherein the composition is formulated as a food product.

8. The method as claimed in claim 1, wherein the composition is formulated as a pharmaceutical composition.

9. The method as claimed in claim 8, wherein the pharmaceutical composition is in a dosage form selected from the group consisting of a parenteral dosage form, an oral dosage form, a topical dosage form, and an inhalation dosage form.

10-25. (canceled)
